SEQUENCE LISTING (10 sequences)

SEQ ID NO: 1 - DNA, 1737 bp, synthetic construct
  misc_feature 1..1737: note = sequence of mutated gE gene encoding wild-type PRV
  source 1..1737: mol_type = other DNA
acgcggccctttctgctgcgcgccgcgcagctcctggcgctgctggccctggcgctctccaccgaggccccgagcctctccgccgagacgaccccgggccccgtcaccgaggtcccgagtccctcggccgaggtctgggacgacctctccaccgaggccgacgacgatgacctcaacggcgacctcgacggcgacgaccgccgcgcgggcttcggctcggccctcgcatccctgagggaggcgcccccggcccatctggtgaacgtgtccgagggcgccaacttcaccctcgacgcgcgcggcgacggcgccgtgctggccgggatctggacgttcctgcccgtccgcggctgcgacgccgtgtcggtgaccacggtgtgcttcgagaccgcgtgccacccggacctggtgctgggccgcgcctgcgtccccgaggccccggagatgggcatcggcgactacctgccgcccgaggtgccgcggctccggcgcgagccgcccatcgtcaccccggagcggtggtcgccgcacctgagcgtcctgcgggccacgcccaacgacacgggcctctacacgctgcacgacgcctcggggccgcgggccgtgttctttgtggcggtgggcgaccggccgcccgcgccggcggacccggtgggccccgcgcgccacgagccccgcttccacgcgctcggcttccactcgcagctcttctcgcccggggacacgttcgacctgatgccgcgcgtggtctcggacatgggcgactcgcgcgagaactttaccgccacgctggactggtactacgcgcgcgcgcccccgcggtgcctgctgtactacgtgtacgagccctgcatctaccacccgcgcgcgcccgagtgcctgcgcccggtggacccggcgtgcagcttcacctcgccggcgcgcgcgcggctggtggcgcgccgcgcgtacgcctcgtgcagcccgctgctcggggaccggtggctgaccgcctgccccttcgacgccttcggcgaggaggtgcacacgaacgccaccgcggacgagtcggggctgtacgtgctcgtgatgacccacaacggccacgtcgccacctgggactacacgctcgtcgccaccgcggccgagtacgtcacggtcatcaaggagctgacggccccggcccgggccccgggcaccccgtggggccccggcggcggcgacgacgcgatctacgtggacggcgtcacgacgccggcgccgcccgcgcgcccgtggaacccgtacggccggacgacgcccgggcggctgtttgtgctggcgctgggctccttcgtgatgacgtgcgtcgtcgggggggccatctggctctgcgtgctgtgctcccggcgccgggcggcctcgcggccgttccgggtgccgacgcgggcgcggacgcacatgctctctccggtgtacaccagcctgcccacgcacgaggactactacggcgacgacgacgacgacgaggaggcgggcgtcatccgccggcggcccgcctcccccagcggagacagcggctacgaggggccgtacgcgagcctggaccccgaggacgagttcagcagcgacgaggacgacgggctgtacgtgcgccccgaggaggcgccccgctccggcttcgacgtctggttccgcgatccggagaaaccggaagtgacgaatggacccaactatggcgtgaccgccaaccgcctgttgatgtcccgccccgcttaa

SEQ ID NO: 2 - DNA, 20 bp, synthetic construct
  misc_feature 1..20: note = sgRNA sequence
  source 1..20: mol_type = other DNA
ggccgcatggtctcaacccc

SEQ ID NO: 3 - DNA, 24 bp, synthetic construct
  misc_feature 1..24: note = forward sequence of single-stranded oligonucleotide NG-ABE8e-gE-F
  source 1..24: mol_type = other DNA
caccggccgcatggtctcaacccc

SEQ ID NO: 4 - DNA, 24 bp, synthetic construct
  misc_feature 1..24: note = reverse sequence of single-stranded oligonucleotide NG-ABE8e-gE-R
  source 1..24: mol_type = other DNA
aaacggggttgagaccatgcggcc

SEQ ID NO: 5 - DNA, 19 bp, synthetic construct
  misc_feature 1..19: note = sequence of forwar primer gE-T-F for PCR detection
  source 1..19: mol_type = other DNA
tgatcttcctgggcgggat

SEQ ID NO: 6 - DNA, 19 bp, synthetic construct
  misc_feature 1..19: note = sequence of reversxe primer gE-T-R for PCR detection
  source 1..19: mol_type = other DNA
tcggacacgttcaccagat

SEQ ID NO: 7 - DNA, 26 bp, synthetic construct
  source 1..26: mol_type = other DNA; note = Original virus (WT-PRV)
gtaccggcgtcgatgatgatggtggc

SEQ ID NO: 8 - DNA, 26 bp, synthetic construct
  source 1..26: mol_type = other DNA; note = Edit NG-ABE8e gene
gtaccggcgtcgacgatgatggtggc

SEQ ID NO: 9 - DNA, 28 bp, synthetic construct
  source 1..28: mol_type = other DNA; note = NG-ABE8e gene of original virus
gggttgagaccatgcggccctttctgct

SEQ ID NO: 10 - DNA, 28 bp, synthetic construct
  source 1..28: mol_type = other DNA; note = NG-ABE8e gene of edit virus
gggttgagaccacgcggccctttctgct

(19) United States
(12) Patent Application Publication
(10) Pub. No.: US 2025/0136951 A1
(43) Pub. Date: May 1, 2025

(54) METHOD FOR CONSTRUCTING gE GENE-DELETED PSEUDORABIES VIRUS STRAIN USING ADENINE BASE EDITOR AND USE THEREOF

(71) Applicant: JIANGXI AGRICULTURAL UNIVERSITY, Nanchang City, Jiangxi Province (CN)

(72) Inventors: Yu YE, Nanchang City, Jiangxi Province (CN); Peixia WANG, Nanchang City, Jiangxi Province (CN); Yuxin TANG, Nanchang City, Jiangxi Province (CN); Chuan ZENG, Nanchang City, Jiangxi Province (CN); Jun GU, Nanchang City, Jiangxi Province (CN); Jinyan SHEN, Nanchang City, Jiangxi Province (CN); Yiwen DUAN, Nanchang City, Jiangxi Province (CN); Yuwei BAI, Nanchang City, Jiangxi Province (CN); Dongyan HUANG, Nanchang City, Jiangxi Province (CN); Deping SONG, Nanchang City, Jiangxi Province (CN)

(73) Assignee: JIANGXI AGRICULTURAL UNIVERSITY, Nanchang City, Jiangxi Province (CN)

(21) Appl. No.: 18/903,289

(22) Filed: Oct. 1, 2024

(30) Foreign Application Priority Data
Oct. 25, 2023 (CN) 202311385572.1

(51) Int. Cl. C12N 7/04; C12N 15/10

(52) U.S. Cl. CPC C12N 7/045; C12N 15/1031; C12N 2710/16721

(57) ABSTRACT

Provided is a method for constructing a gE gene-deleted pseudorabies virus (PRV) strain using an adenine base editor (ABE) and use thereof. The method includes: designing an sgRNA sequence using the ABE with a start codon of the gE gene in a PRV as a target site, ligating an enzyme-digested fragment to a double-stranded DNA fragment with sticky ends to obtain a ligation product; and transforming the ligation product into a competent cell to allow plate screening and culture, selecting a resulting positive bacterial strain to allow expanded culture, and extracting a plasmid from a resulting positive bacterial solution; and transferring the plasmid into a target cell to allow the transfection for 24 h, collecting a resulting virus liquid, and centrifuging the virus liquid to collect a resulting supernatant.

### CROSS-REFERENCE TO RELATED APPLICATION

This patent application claims the benefit and priority of Chinese Patent Application No. 202311385572.1 filed with the China National Intellectual Property Administration on Oct. 25, 2023, the disclosure of which is incorporated by reference herein in its entirety as part of the present application.

### REFERENCE TO SEQUENCE LISTING

A computer readable XML file entitled “HLP20240100365-sequence listing”, which was created on Aug. 8, 2024, with a file size of about 10,701 bytes, contains the sequence listing for this application, has been filed with this application, and is hereby incorporated by reference in its entirety.

### TECHNICAL FIELD

The present disclosure relates to the field of biotechnology, specifically to a method for constructing a gE gene-deleted pseudorabies virus (PRV) strain using an adenine base editor (ABE) and use thereof.

### BACKGROUND

Pseudorabies is a highly contagious, septicemic, and severe infectious disease caused by pseudorabies virus (PRV) infection. Pigs of all ages are susceptible to the PRV. Pregnant sows infected with PRV can cause abortion, stillbirth, mummification, or weak fetuses. Infection of piglets with PRV can cause high fever, neurological symptoms, and dyspnea, while newborn piglets mostly show neurological symptoms after infection. After breeding pigs are infected with the PRV, breeding pigs may be infertile, sows may not be in estrus to cause difficulty in breeding, and boar testicles may be swollen and atrophied, losing the ability to breed. PRV is a double-stranded DNA virus with a full genome length of about 150 kb and an average G+C content of up to 74%. The PRV consists of a unique long segment (UL), a unique short segment (US), terminal repeats (TRs) on both sides, and internal repeats (IRs). Studies have found that gE is the main virulence gene but is not required for virus proliferation. Therefore, silencing the virulence gene gE can reduce the virulence of virus without affecting the immunogenicity, such that a resulting attenuated virus strain can be used as a vaccine candidate strain for the control and eradication of pseudorabies.

Early gene editing technologies generally adopt a principle of DNA homologous recombination, including zinc-finger nucleases (ZFNs) and transcription activator-like effector nucleases (TALENs), but their development was limited due to shortcomings such as complex design, low knockout efficiency, serious off-target, high cost, and poor operability. Since its discovery, the clustered regularly interspaced short palindromic repeats/CRISPR-associated protein 9 (CRISPR/Cas9) system has become the most popular genetic modification tool due to its efficiency, convenience, and wide range of applications, achieving successful gene knockout. However, similar to technologies such as ZFNs and TALENs, gene editing by CRISPR/Cas9 relies on the generation of DNA double-strand breaks, triggering repair and resulting in unexpected base mutations or off-target cuts and structural variations in the genome. To overcome this shortcoming, the emergence of single base editing technology brings hope for achieving precise and efficient base conversion.

The single base editing technology is to fuse a modified Cas protein with no nuclease cleavage activity or single-strand nicking activity and a deaminase, and accurately anchor a resulting fused deaminase to a target site with sgRNA to allow base deamination, thereby achieving precise editing of single bases. This technology greatly improves the accuracy and efficiency of base editing. tRNA adenylate deaminase (TadA) is fused with nCas9 to develop a novel single-base conversion system (namely an adenine base editor, ABE) that can accurately convert adenine into guanine. After 7 rounds of transformation, a higher-efficiency ABE version ABE7.10 is selected, with its effective editing window at positions 4 to 7 of the sgRNA. A deaminase component of the ABE7.10 continues to improve, resulting in a current version of the ABE with a higher editing efficiency: ABE8e. Compared with ABE7.10, the ABE8e contains 8 additional mutations, which can increase the activity 590 times, and can greatly improve the editing efficiency when being paired with various Cas9 or Cas12 homologues. Since the development of ABE tools, studies have confirmed that the single base editors can work efficiently in multiple species, such as bacteria, plants, and mammals, but their application in viral genome modification has not yet been reported.

Existing ZFNs and TALENs are time-consuming, labor-intensive, and expensive to use; while the CRISPR/Cas9 shows low efficiency, difficulty in screening, and the generation of structural variations due to genome breaks. Although other base editing tools can achieve silencing, more bystander effects are generated and some areas cannot be edited due to PAM restrictions.

### SUMMARY

In order to solve at least one of the technical problems existing in the prior art, a purpose of the present disclosure is to provide a method for constructing a gE gene-deleted PRV strain using an ABE and use thereof.

The technical solutions of the present disclosure are as follows:

The present disclosure provides a gE gene-deleted PRV strain, where the gE gene-deleted PRV strain is deposited in the China Center for Type Culture Collection (CCTCC) on Jun. 30, 2023, in the Wuhan University, Wuhan City, Hubei Province, China, with a deposit number of CCTCC NO: V202323; and the gE gene-deleted PRV strain is named as porcine pseudorabies virus PRV-AgE-ABE and constructed using an ABE.

Preferably, a base at position 2 of a start codon in the gE gene of wild-type PRV is mutated from T to C, and a resulting mutated gE gene is set forth in SEQ ID NO: 1.

The present disclosure further provides a method for constructing the gE gene-deleted PRV strain using an ABE, including the following steps:

S1, constructing an sgRNA backbone plasmid:
designing an sgRNA sequence using the ABE with a start codon of the gE gene in a PRV as a target site, synthesizing a single-stranded oligonucleotide according to the sgRNA sequence, annealing the single-stranded oligonucleotide to obtain a double-stranded DNA fragment with sticky ends, digesting a pU6-sgRNA-Puro-2A-EGFP vector with a restriction endonuclease and recovering a resulting enzyme-digested fragment, and then ligating the enzyme-digested fragment to the double-stranded DNA fragment with sticky ends to obtain a ligation product, namely an sgRNA expression vector; and
transforming the ligation product into a competent cell to allow plate screening and culture, selecting a resulting positive bacterial strain to allow expanded culture, and extracting a plasmid from a resulting positive bacterial solution; and
S2, transfection of the plasmid:
transferring the plasmid into a Vero81 cell to allow the transfection, collecting a resulting virus liquid, and centrifuging the virus liquid to collect a resulting supernatant.

Preferably, the sgRNA sequence is set forth in SEQ ID NO: 2.

Preferably, the restriction endonuclease is a BbsI enzyme.

Preferably, the Vero81 cell is cultured in a fetal bovine serum (FBS)-containing medium to a logarithmic growth phase before the transfection is conducted.

Preferably, the single-stranded oligonucleotide has a sequence as follows:

NG-ABE8e-gE-F:
(SEQ ID NO: 3)
CACCGGCCGCATGGTCTCAACCCC;
NG-ABE8e-gE-R:
(SEQ ID NO: 4)
AAACGGGGTTGAGACCATGCGGCC.

Preferably, NG-ABE8e is a plasmid expressing the nCas9 protein.

The present disclosure further provides a gE gene-deleted PRV strain prepared by the method.

The present disclosure further provides use of the gE gene-deleted PRV strain in preparation of a pseudorabies vaccine.

The present disclosure has the following beneficial effects:

In the present disclosure, the ABE is used to modify the start codon ATG with reference to FIG. 1, and can block protein translation from the source. The method can be used as a new gene silencing technology to supplement the limitations of editing sites in the prior art. In the gE gene-deleted PRV strain prepared using this method, there is no structural variation in the PRV strain, and the termination of gE expression can be achieved by simply modifying a small amount of A or T bases in ATG. Moreover, a potential regulatory role of the gene sequence is minimally affected.

In the present disclosure, the method has high efficiency and small changes to viral genes, does not cause large structural variations in the viral genome, and is low in cost.

In the present disclosure, the gE gene-deleted PRV strain is deposited in the CCTCC on Jun. 30, 2023, in the Wuhan University, Wuhan City, Hubei Province, China, with a deposit number of CCTCC NO: V202323; and the gE gene-deleted PRV strain is named: porcine pseudorabies virus PRV-AgE-ABE.

### BRIEF DESCRIPTION OF THE DRAWINGS

FIG. 1 shows a principle flow chart for constructing the gE gene-deleted PRV strain in the present disclosure;

FIG. 2 shows a nucleic acid electrophoresis gel image of the PCR product after mutation of a first start codon of the gE gene in the present disclosure; where lane M represents a DL2000 Marker, and lane 1 represents a target band for sequencing;

FIG. 3 shows a sequencing map of the viral gE gene edited in the present disclosure; where the underline represents an expected mutant gene, the triangle represents a mutation site, and the arrow represents a result after mutation;

FIG. 4 shows a sequencing profile of PRV-AgE after 10 passages; and

FIG. 5 shows expression verification results of a gE protein of the PRV-AgE strain.

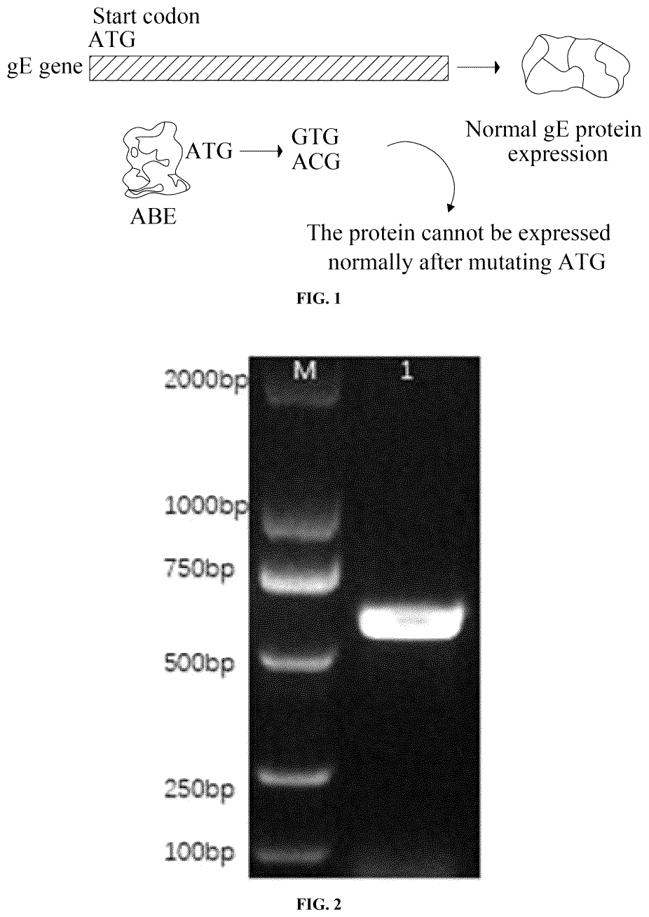

### DETAILED DESCRIPTION OF THE EMBODIMENTS

The technical solutions of the present disclosure will be further described in detail below in conjunction with specific examples.

All experimental materials used in the following examples are purchased from conventional biochemical reagent companies, unless otherwise specified.
NG-ABE8e vector: Addgene, product number: 138491;
in the NG-ABE8e vector, a CMV promoter drives the expression of related proteins;
an ABE NG-ABE8e expresses adenine deaminase for point mutations; and
an sgRNA backbone plasmid includes a binding region that binds the target sequence and a binding region that binds the Cas9 protein.
   I. Construction of an sgRNA Backbone Plasmid

In this example, a start codon of the gE gene was selected as a target site;

An sgRNA with a recognition sequence was designed as follows:

NG-ABE8e-gE:
(SEQ ID NO: 2)
GGCCGCATGGTCTCAACCCC;

A single-stranded oligonucleotide was synthesized according to the designed sequence. A sequence of the single-stranded oligonucleotide was as follows:

NG-ABE8e-gE-F:
(SEQ ID NO: 3)
CACCGGCCGCATGGTCTCAACCCC;
NG-ABE8e-gE-R:
(SEQ ID NO: 4)
AAACGGGGTTGAGACCATGCGGCC;

the NG-ABE8e-gE-F and NG-ABE8e-gE-R were annealed to obtain a double-stranded DNA fragment with sticky ends;
a pU6-sgRNA-Puro-2A-EGFP vector was digested with a BbsI enzyme, and a resulting fragment was recovered and then ligated to the double-stranded DNA fragment with sticky ends to obtain an sgRNA expression vector; and
an ABE NG-ABE8e included Cas9 (N), which in this example could specifically be a plasmid expressing the nCas9 protein.
   (1) Construction of a Double-Stranded sgRNA

The following table showed a phosphorylation annealing system;

TABLE 1
Phosphorylation annealing system

| Reagent | System (μL) |
|---|---|
| Forward single strand (100 μM) | 1 |
| Reverse single strand (100 μM) | 1 |
| 10 × T4 DNA ligation buffer (NEB) | 1 |
| T4 PNK (NEB) | 1 |
| Double distilled water | Supplementing to 10 μL |

A gradient annealing program was shown in Table 2:

TABLE 2
Gradient annealing program

| Temperature | Time | Gradient |
|---|---|---|
| 37° C. | 30 min | — |
| 95° C. | 5 min | — |
| 95-85° C. | 1 min | −5° C. |
| 85-75° C. | 1 min | −5° C. |
| 75-65° C. | 1 min | −5° C. |
| 65-55° C. | 1 min | −5° C. |
| 55-45° C. | 1 min | −5° C. |
| 45-35° C. | 1 min | −5° C. |
| 35-25° C. | 1 min | −5° C. |
| 4° C. | Hold | — |

### (2) Construction and Expression of a Linearized Backbone Vector Plasmid

The table below (Table 3) showed an enzyme digestion system of PX459 vector.

TABLE 3
Enzyme digestion system of PX459 vector

| Reagent | Volume (μL) |
|---|---|
| 10 × NEB | 5 |
| Restriction endonuclease BbsI | 1 |
| PX459 vector (2 μg) | 3.5 |
| Double distilled water | 40.5 |
| Total | 50 |

Reaction procedure: the enzyme digestion system was placed in a 37° C. water bath for 2 h to obtain an enzyme digestion product.

The enzyme digestion product was detected by agarose gel electrophoresis, and a target band was recovered from a gel the after electrophoresis.

(3) Construction of a Backbone Plasmid Expressing sgRNA

The following table (Table 4) showed a ligation reaction system.

TABLE 4
Ligation reaction system

| Reagent | Volume (μL) |
|---|---|
| 10 × T4 DNA ligation buffer | 1 |
| T4 DNA ligase (NEB) | 1 |
| Linearized PX459 vector (50 ng) | 2 |
| 200-fold-diluted gE-sgRNA annealing product | 1 |
| Double distilled water | 5 |
| Total | 10 |

The ligation product was transformed into Top10 competent cells by conventional methods. An appropriate amount of resulting bacterial solution was applied onto an LB plate containing ampicillin. After liquid on the surface of the plate evaporated, the plate was put upside down in a 37° C. incubator to allow overnight culture for 16 h to 18 h, and the transformation was observed. 5 single-clonal colonies were selected, cultured with shaking and sequenced. The positive colonies with correct sequencing were further selected to allow expanded culture, and endotoxic-free plasmids for cell transfection were extracted and then stored at −20° C.

### II. Plasmid Transfection

(1) One day before transfection, the well-growing Vero81 cells were digested with trypsin, 1×10⁶ digested cells were transferred to a six-well plate, and added with 2 mL of DMEM medium containing 10% (v/v) FBS (purchased from Gibco), and cultured to logarithmic growth phase at 37° C. and 5% (v/v) CO₂.
(2) Cell transfection was conducted according to the official instructions of Lipofectamine3000, where a total amount of transfected DNA was 3 μg, including 1 μg each of viral genomic DNA, NG-ABE8e plasmid, and sgRNA expression vector plasmid.
(3) 24 h after transfection, the presence or absence of PRV characteristic lesions was observed under a microscope. After lesions were observed, the six-well plate was frozen and thawed three times at −80° C. to room temperature, the collected virus liquid was centrifuged at 8,000 rpm for 5 min, and a portion of the collected supernatant was used to extract genes for sequencing. The detection primers were synthesized according to Table 5, and the target genes were amplified using the corresponding primers. The procedures were shown in Table 6. During amplification, a temperature with the lowest Tₘ value in the primer pair was used as an annealing temperature of the PCR reaction, and the extension time was set to 7 s according to the characteristics of the polymerase used. The results of PCR products were analyzed by agarose gel electrophoresis, and the successfully identified target fragments were cut out and sent to Shanghai Sangon Biotech Co., Ltd. for sequencing.

TABLE 5
Synthetic sequences of PCR detection primers

| | | Target |
|---|---|---|
| | | fragment |
| Name | Sequence (5′-3′) | size |
| gE-T-F | TGATCTTCCTGGGCGGGAT | 584 bp |
| | (SEQ ID NO: 5) | |
| gE-T-R | TCGGACACGTTCACCAGAT | |
| | (SEQ ID NO: 6) | |

TABLE 6
PCR program

| | Temperature (° C.) | Time | |
|---|---|---|---|
| Initial denaturation | 98 | 30 s | |
| Denaturation | 98 | 10 s | 35 cycles |
| Annealing | 57 | 5 s | 35 cycles |
| Extension | 72 | 7 s | 35 cycles |
| Total extension | 72 | 1 min | |
| Short-term storage | 4 | 30 min | |

Results: PCR detection was conducted using gE-T-F and gE-T-R as upstream and downstream primers, and a target band of the expected size was obtained. The results of gel electrophoresis of PCR products are shown in FIG. 2.
(4) After successful sequencing, the sequencing results were compared with the wild-type PRV gene to obtain detailed mutation information.

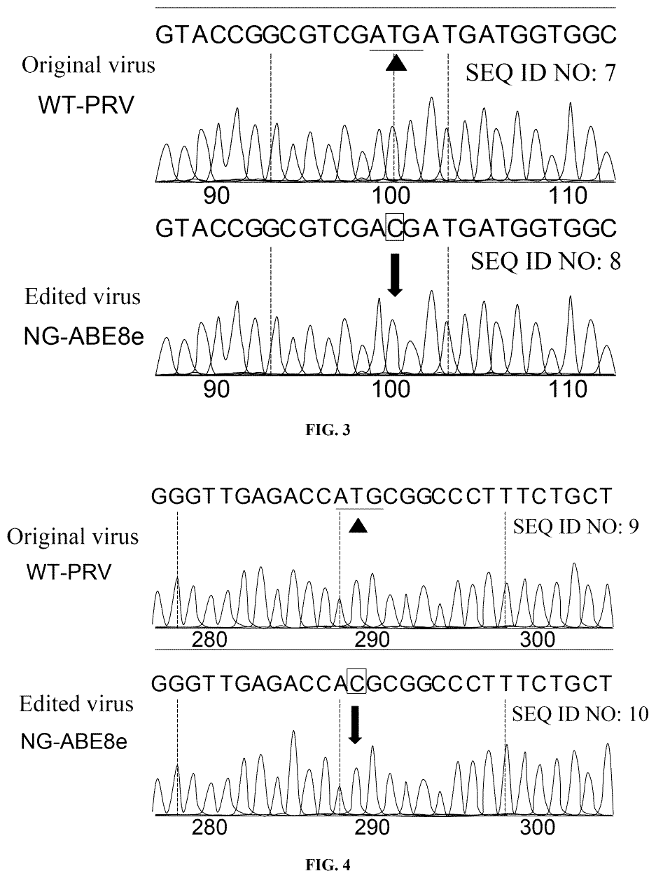

The sequencing results are shown in FIG. 3. The comparison results showed that base T was successfully mutated into base C, with only a small amount of T remaining. A mutated gE gene was set forth in SEQ ID NO: 1.

The obtained gE gene-deleted PRV strain was deposited in the CCTCC on Jun. 30, 2023, with a deposit number of CCTCC NO: V202323; and the gE gene-deleted PRV strain was named: porcine pseudorabies virus PRV-AgE-ABE.

### III. Plaque Purification of Mutant Strain

(1) The virus supernatant was serially diluted 10 to 10⁶ times in a ten-fold ratio. The different dilutions of virus liquids were inoculated separately into a 12-well plate filled with Vero81 monolayer cells, with 200 μL in each well, the cells were incubated for 2 h with the cell plate shaken every half hour. The virus liquid was discarded, and cell surface was washed 3 times with a serum-free DMEM medium for later use.
(2) The autoclaved 2% (w/v) low melting point agarose was dissolved by microwave heating, cooled to about 37° C., mixed thoroughly with an equal volume of 2×DMEM medium containing 4% FBS, quickly added into the 12-well plate, with 1 mL to each well, and placed at room temperature for about 0.5 h. After the medium solidified naturally, the plate was put upside down in a carbon dioxide cell incubator for culture.
(3) After 48 h of incubation, cell lesions and plaque formation were observed. Multiple single plaques were randomly selected with a 10 μL pipette tip in the low dilution wells, blown into 200 μL of DMEM medium, mixed well and inoculated with Vero81 cells in a 24-well plate to allow expanded culture.
(4) After a large number of lesions appeared on the cells, the virus liquid was collected, the viral DNA was extracted, the target fragment was amplified using primers, followed by proceeding to the next round of purification based on the corresponding sequencing results.
(5) After purification, the virus was passed through 10 consecutive passages, sequencing was conducted to verify the stability of the mutant gene, and the virus liquid was collected.

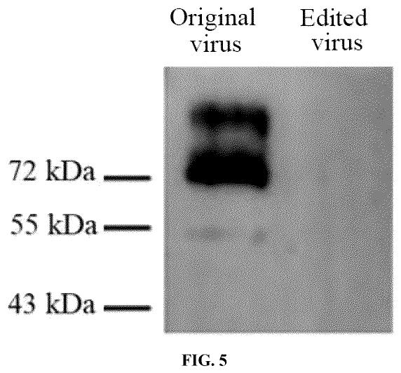

Results: the PRV-ΔgE-ABE gene sequencing results after 10 passages were shown in FIG. 4. Based on the sequencing results, it was preliminarily determined that the inheritance of single-base mutations in the gE gene was relatively stable.

### IV. Analysis of Expression of Viral Proteins Caused by Gene Silencing

(1) Whether the expression of target gene was silenced detected by Western blot

The purified virus was internalized into Vero81 cells, and the viral protein was collected 24 h after inoculation, and Western blot was conducted to detect the expression of the gE gene.
(2) The experimental results are shown in FIG. 5. The results showed that compared with the wild-type strain, the expression of gE protein was not detected in the mutated gE gene, indicating that mutating the start codon could effectively inhibit the expression of gE protein.
(3) The above results showed that using the NG-ABE base editor to mutate the start codon of the gene could inhibit the expression of gE in the virus, thereby achieving better gene silencing effects.

The above embodiments are merely illustrative of some implementations of the present disclosure, and the description thereof is specific and detailed, but should not be construed as limiting the patent scope of the present disclosure. Corresponding changes and variations may be made by those skilled in the art according to the technical solutions and concepts described above, and all these changes and variations should fall within the protection scope of the claims of the present disclosure.

What is claimed is:

1. A gE gene-deleted pseudorabies virus (PRV) strain, wherein the gE gene-deleted PRV strain is deposited in the China Center for Type Culture Collection (CCTCC) with a deposit number of CCTCC NO: V202323; and the gE gene-deleted PRV strain is constructed using an adenine base editor (ABE);
wherein a base at position 2 of a start codon in the gE gene of wild-type PRV is mutated from T to C, and a resulting mutated gE gene is set forth in SEQ ID NO: 1; and
wherein the gE gene-deleted PRV strain is constructed using the following steps:
S1, constructing an sgRNA backbone plasmid, comprising:
designing an sgRNA sequence using the ABE with a start codon of the gE gene in a PRV as a target site, synthesizing a single-stranded oligonucleotide according to the sgRNA sequence, annealing the single-stranded oligonucleotide to obtain a double-stranded DNA fragment with sticky ends, digesting a pU6-sgRNA-Puro-2A-EGFP vector with a restriction endonuclease and recovering a resulting enzyme-digested fragment, and then ligating the enzyme-digested fragment to the double-stranded DNA fragment with sticky ends to obtain a ligation product, namely an sgRNA expression vector; and
transforming the ligation product into a competent cell to allow plate screening and culture, selecting a resulting positive bacterial strain to allow expanded culture, and extracting a plasmid from a resulting positive bacterial solution; and
S2, transfection of the plasmid, comprising:
transferring the plasmid into a Vero81 cell to allow the transfection, collecting a resulting virus liquid, and centrifuging the virus liquid to collect a resulting supernatant;
wherein the adenine base editor is NG-ABE8e; and
the sgRNA sequence is set forth in SEQ ID NO: 2.

2. A method for constructing the gE gene-deleted PRV strain according to claim 1 using an ABE, comprising the following steps:
S1, constructing an sgRNA backbone plasmid, comprising:
designing an sgRNA sequence using the ABE with a start codon of the gE gene in a PRV as a target site, synthesizing a single-stranded oligonucleotide according to the sgRNA sequence, annealing the single-stranded oligonucleotide to obtain a double-stranded DNA fragment with sticky ends, digesting a pU6-sgRNA-Puro-2A-EGFP vector with a restriction endonuclease and recovering a resulting enzyme-digested fragment, and then ligating the enzyme-digested fragment to the double-stranded DNA fragment with sticky ends to obtain a ligation product, namely an sgRNA expression vector; and
transforming the ligation product into a competent cell to allow plate screening and culture, selecting a resulting positive bacterial strain to allow expanded culture, and extracting a plasmid from a resulting positive bacterial solution; and
S2, transfection of the plasmid, comprising:
transferring the plasmid into a Vero81 cell to allow the transfection, collecting a resulting virus liquid, and centrifuging the virus liquid to collect a resulting supernatant.

3. The method according to claim 2, wherein the restriction endonuclease is a BbsI enzyme.

4. The method according to claim 2, wherein the Vero81 cell is cultured in a fetal bovine serum (FBS)-containing medium to a logarithmic growth phase before the transfection is conducted.
